(19) United States
(12) Patent Application Publication
(10) Pub. No.: US 2008/0312698 A1
(43) Pub. Date: Dec. 18, 2008

(54) DEVICE AND SYSTEM FOR STABILIZING MOVEMENT BETWEEN BONY TISSUE AND METHOD FOR IMPLANTING

(76) Inventors: BRIAN J. BERGERON, Austin, TX (US); Charles R. Forton, Leander, TX (US); Jeremy J. Lemoine, Austin, TX (US); Jamie R. Murdock, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/763,268

(22) Filed: Jun. 14, 2007

(51) Int. Cl. A61B 17/70; A61B 17/58; A61B 17/56; A61B 17/04; A61F 2/44

(52) U.S. Cl. 606/280; 623/17.16; 606/70; 606/301

(57) ABSTRACT

A device and system for stabilizing movement between two or more vertebral bodies and methods for implanting. Specifically, embodiments of the present disclosure may provide medical professionals the ability to selectively position and orient anchors in bony tissue and then attach a plate to the pre-positioned anchors. The plate assembly, once positioned on the anchors, prevents the anchors from backing out of the bony tissue.

### TECHNICAL FIELD OF THE DISCLOSURE

This disclosure relates generally to stabilizing movement between bony tissues within a body, and in particular to systems and methods for stabilizing movement between vertebral bodies. Even more particularly, embodiments of the present disclosure relate to systems and methods for stabilizing movement between vertebral bodies in the cervical portion of the spine.

### BACKGROUND OF THE DISCLOSURE

In medical situations, it is sometimes necessary to limit or eliminate movement between bony tissues such as vertebral bodies, either temporarily or permanently. As an example, after an accident, it may be necessary to temporarily limit the movement of vertebrae to prevent possible damage to the spinal cord that could lead to paralysis. As another example, during treatment for a degenerative disk, permanent fusion of two or more adjacent vertebral bodies may be necessary to alleviate pain or preserve some functionality.

Prior art systems generally require that a plate be positioned over the vertebral bodies and then positioned using multiple screws on each vertebral body. The term anchors, as used throughout this document, generally refers to a device configured with one end having a helical thread configured to penetrate and remain in bony tissue, and a second end configured, such as with a key-style profile, internal/external hex, or other design, to enable a surgeon to rotate the anchor to engage the threads in the bony tissue at a desired site. However, these methods and systems are undesirable because of the obstacles to overcome when implanting the plate and the subsequent risk that screws holding the plate in place will back out.

A disadvantage with implanting prior art systems involves lining up the plate with the vertebral bodies. When stabilizing a cervical spine, for example, a surgeon is likely to access the point from an angle lateral to the patient because the trachea, along with muscles and tissues, prevent direct anterior access to the cervical spine. This can make it difficult to ensure that the plate is aligned to the satisfaction of the surgeon.

Somewhat related to the problem of alignment is the difficulty of anchoring the plate in place. The difficulty of anchoring the plate may be the result of at least two related problems: the use of multiple screws and positioning of the screws. Prior art systems generally rely on multiple screws to stabilize a vertebral body to a vertebral body. However, multiple screws may be undesirable or even contraindicated in certain situations. Positioning the screws is difficult due to the lateral approach cited above, as well as the need for extensive retraction. In particular, prior art systems also generally have plates with holes located on either side of midline. The difficulty lies when attempting to implant screws into the plate on the far lateral side of midline because of the retraction needed to access these areas. Retraction of nearby muscles, tissues, and organs might be a necessary component of implanting a stabilization system. However, it is common knowledge to medical professionals that reducing the stress or damage done to the body reduces healing time and the risk of other complications.

In addition to the stress or damage that retraction may cause to the body, retraction has other limits. For example, when retracting tissue to access the cervical spine, the trachea must be retracted. Because the trachea is limited in how far it can be retracted, it creates an anatomical boundary. Surgeons implanting screws at points past midline are therefore restricted in how they can access these points, making the surgery more difficult.

Once a plate has been implanted into the body, there is also the concern that a screw will back out of the plate, resulting in a dangerous situation in which the plate might not be able to stabilize the movement, and the screw might damage internal organs or tissues.

### SUMMARY OF THE DISCLOSURE

Embodiments of the present disclosure provide devices, systems and methods for stabilizing movement between bony tissues in a body, using fewer anchors, preventing screws from backing out, and causing less damage and stress to the body. One embodiment includes a plate assembly that allows medical professionals to implant anchors into the bony tissue and then attach the plate assembly to the anchors. In this embodiment, a plate assembly is configured to capture a portion of a first anchor on a first bony tissue and a portion of a second anchor located in a second bony tissue, and is further configured to prevent the first anchor from backing out of the first bony tissue and the second anchor from backing out of the second bony tissue. In some embodiments, the plate assembly is configured with an attachment feature such as a track. In some embodiments the plate has a first end with a recessed portion on a first edge shaped to receive the portion of the first anchor and a second end with a recessed portion on a second edge obverse from the first edge shaped to receive the portion of the second anchor, and the plate is configured to capture the first and second anchors in the recessed portions by rotating the plate assembly from a first orientation to a second orientation. In other embodiments, the present disclosure comprises a first plate configured for selected contact with a plurality of anchors and a second plate configured for selected contact with the plurality of anchors wherein the second plate is configured for secure connection to the first plate to capture the plurality of anchors. The first plate may connect to the second plate using a slidable connector, a rotatable connector, or a mated connector such as a ratcheting mechanism or a screw mechanism. In some embodiments, the first and second plates are configured with one or more recessed portions such as a plurality of notches or a groove, to capture one or more anchors. The recessed portions may have a layer with a desired friction coefficient for providing a desired range of movement. The plate assembly may be manufactured from biocompatible or resorbable material, and may further include a spacer for implantation between two vertebral bodies.

Another embodiment of the present disclosure includes a system for stabilizing the movement between bony tissues in body, using a first anchor implantable in a first selected bony tissue, a second anchor implantable in a second selected bony tissue, and a plate assembly configured to capture a portion of the first anchor and a portion of the second anchor, and further configured to prevent the first anchor from backing out of the first bony tissue and the second anchor from backing out of the second bony tissue. The plates and anchors may be manufactured from biocompatible material or resorbable material. The anchors may be screws with standard features or may have spacers, flanges, spherical heads, angled tapers, polyaxial joints, or other features to facilitate implantation of the anchor or subsequently preventing the anchor from backing out or being damaged over time. Once the plates capture the anchors, the plate assembly may prevent the anchors from backing out of the bony tissue.

Yet another embodiment of the present disclosure is directed to a method for stabilizing movement between bony tissue by implanting a first anchor in a selected location on a first bony tissue, implanting a second anchor in a selected location on a second bony tissue, and capturing a portion of the first anchor and a portion of the second anchor, by a plate assembly configured to capture the first anchor and the second anchor, wherein the plate assembly is further configured to prevent the first anchor from backing out of the first bony tissue and the second anchor from backing out of the second bony tissue. In some embodiments, a third anchor is implanted in a selected location on a third bony tissue or on the second bony tissue, and the first, second and third implanted anchors are captured by the plate assembly. Capturing the anchors may be done by engaging a first plate of the plate assembly to a second plate of the plate assembly, or by capturing a first anchor by an attachment feature and then capturing a second anchor by an attachment feature, such as by sliding a track, or by rotating the plate assembly to capture a first and second anchor. In some embodiments, the plate assembly is attached by first locating an attachment point relative to an anatomical landmark, or relative to a plane, such as midline

The present disclosure overcomes prior art methods and systems for stabilizing bony tissue in a body with a plate assembly useful for capturing implanted anchors.

The present disclosure overcomes prior art devices and systems for stabilizing movement between bony tissue by preventing anchors from backing out of the plate assembly.

These, and other, aspects of the disclosure will be better appreciated and understood when considered in conjunction with the following description and the accompanying drawings. The following description, while indicating various embodiments of the disclosure and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE FIGURES

FIG. 1A is an isometric view of one embodiment of a plate assembly for stabilizing movement in a body;

FIG. 1B is an end view of one embodiment of a plate assembly for stabilizing movement in a body;

FIGS. 2A and 2B are top views of one embodiment of a plate assembly for stabilizing movement in a body;

FIG. 2C is an end view of one embodiment of a plate assembly for stabilizing movement in a body;

FIG. 2D is a side view of one embodiment of a plate assembly for stabilizing movement in a body;

FIG. 3A is an isometric view of one embodiment of a plate assembly for stabilizing movement in a body;

FIG. 3B is a cross-sectional view of one embodiment of a system for stabilizing a portion of the spine in a body;

FIG. 4A is an isometric view of one embodiment of a system for stabilizing a portion of the spine in a body;

FIG. 4B is a cross-sectional view of one embodiment of a system for stabilizing a portion of the spine in a body;

FIG. 4C is an isometric view of one embodiment of a system for stabilizing a portion of the spine in a body;

FIG. 5A is a cross-sectional view of one embodiment of a system for stabilizing a portion of the spine in a body; and

FIG. 5B is an isometric view of one embodiment of a system for stabilizing a portion of the spine in a body.

### DETAILED DESCRIPTION OF THE DISCLOSURE

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, additions or rearrangements within the scope of the underlying inventive concept(s) will become apparent to those skilled in the art after reading this disclosure.

Reference is now made in detail to the exemplary embodiments of the disclosure, examples of which are illustrated in the accompanying drawings. Wherever possible, the same reference numbers will be used throughout the drawings to refer to the same or like parts (elements).

The systems and methods of the disclosure may be particularly useful for stabilizing movement in the cervical spine and thus it is in this context that embodiments of the disclosure may be described It will be appreciated, however, that embodiments of the systems and methods of the present disclosure may be applicable for stabilizing movement in other areas of the spine or body.

One of the reasons that embodiments of the present disclosure may be usefully applied to stabilize movement in the body is the ease of implantation and the subsequent security. In cervical spine surgeries in general, surgeons must retract the sterocleidomastoid muscle, the carotid sheath, sternohyoid muscle, omohyoid muscle, sternothyroid muscle, and the trachea to implant a stabilization device or system. These anatomical boundaries make it difficult to access attachment points that are far lateral. Thus, it is desirable to reduce or eliminate the need to implant anchors past midline to reduce the amount of retraction needed to implant a stabilization system. In addition to making implantation easier for the surgeon, reducing the retraction also reduces the general stress on the body and reduces the risk of damage to the above-mentioned tissues and organs.

To achieve these goals, embodiments of the present disclosure enable medical professionals to align and implant a single screw on each vertebral body in a plane before implanting the plates.

More particularly, embodiments of such a plate assembly that may be securely attached after the anchors have been placed allow medical professionals to properly locate the attachment points for a more exact placement. For example, embodiments of the present disclosure allow medical professionals to implant the anchors such that the plate assembly aligns with the midline of the body or based on easily recognizable anatomical landmarks. In some embodiments, fewer anchors result in less time in the operating room and less damage to the bony tissue being stabilized. The combination of fewer anchors and the ability to position the anchors without the plate assembly reduces the retraction of tissues and organs normally required to access the area, thereby reducing the damage and stress to the bony tissue and body in general. Furthermore, embodiments of the present disclosure enable medical professionals to implant screws (or other anchors) without the risk of screws backing out.

Embodiments of a plate assembly may be essentially flat or curved, and may be pre-formed or adapted for modification during implantation to enable medical professionals the ability to selectively stabilize movement. Embodiments may be manufactured from selected materials for a permanent or temporary solution. For example, in some embodiments in which a permanent stabilization is required, the plate assembly may be manufactured from a biocompatible material such as titanium or steel. In other embodiments, a resorbable material is preferred to allow the bony tissue to fuse and the body to resorb the material.

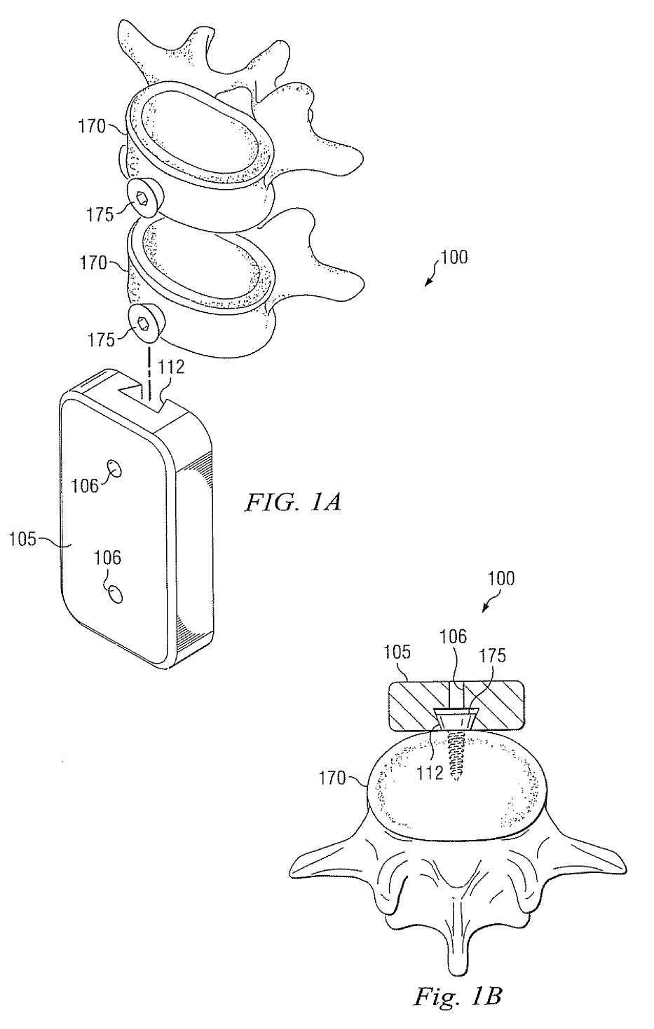

Embodiments of the present disclosure may be better explained with reference to FIGS. 1-5 which depict isometric views of devices and systems for stabilizing movement between vertebral bodies.

FIGS. 1A-B and 2A-D show embodiments of the present disclosure in which plate assembly 100 utilizes a single plate 105 that captures anchors 175 implanted in bony tissue 170 to stabilize movement of a portion of the spine. For purposes of this document, the term capture generally relates to receiving and maintaining an anchor in selected contact. In some embodiments, an anchor may be received by some rectilinear motion of the plate assembly 100, such as by sliding the head of the anchor in a track with a selected profile. In some embodiments, an anchor may be received by some curvilinear motion of the plate assembly 100, such as by rotating two plates about an axis. The anchor may be maintained due to the configuration of one or more devices or portions of the devices, materials, or additional features.

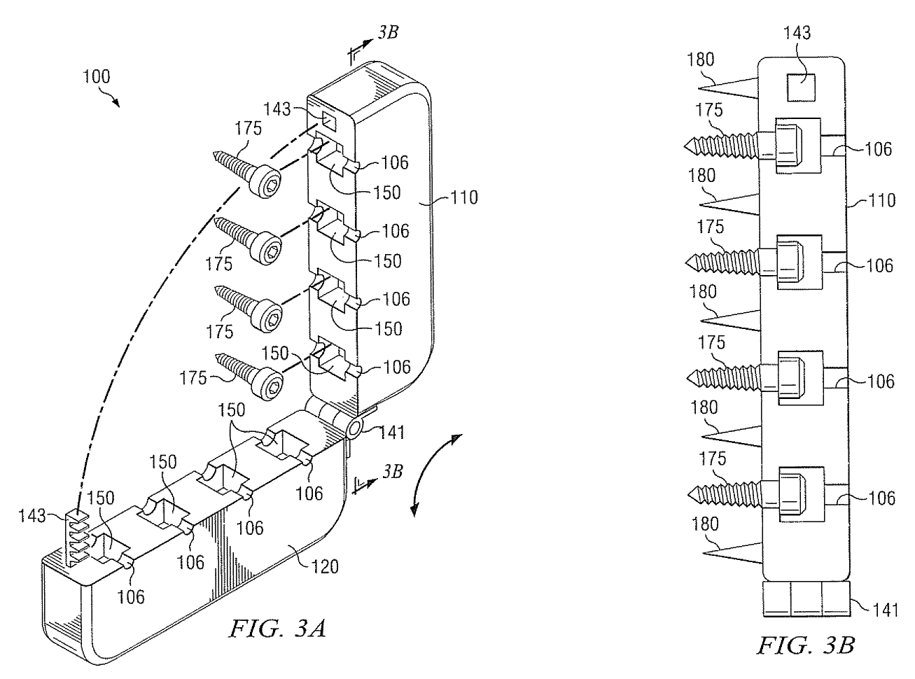

FIGS. 3A-B, 4A-C and 5A-B show embodiments of the present disclosure in which two plates 110 and 120 securely connect to each other to form plate assembly 100 which captures two or more anchors to securely attach to two or more vertebral bodies to stabilize movement in a portion of the spine. In preferred embodiments, a taper, such as a Morse taper, effectively pulls the plate 105 or plate assembly 100 snug to the anchor for added security.

For purposes of this document, the terms stabilize and stabilization refer to the control of one or more degrees of freedom for movement. Rotation, compression, flexion, extension, lateral bending and separation are examples of movement that may need stabilization. Stabilization may result in the complete restriction of movement about a particular axis or plane, or it may limit the movement over a selected range of motion.

For purposes of this document, the terms secure and securely refer to a connection that may be temporary or permanent, but that will not disconnect without assistance from medical professionals. For example, the stabilization system may be a necessary temporary implant after a neck injury. In this type of situation, medical professionals may implant the system to stabilize the neck to prevent further damage, but may want to ensure a minimal range of motion to prevent two vertebral bodies from fusing. Thus, a temporary plate assembly connection that allows the medical professionals to remove the plate assembly at a later date may be preferred. In other situations, for example in which the disk has degenerated and the medical professionals determine that all movement between the disks should be eliminated permanently by fusing the bony tissue together, a permanent secure connection that completely restricts movement may be preferred.

In FIGS. 1A and 1B, plate assembly 100 is shown comprising plate 105 configured, for example with an attachment feature such as a track 112 to capture two or more anchors to stabilize movement between two or more vertebral bodies, and is further advantageously configured to prevent anchors 175 from backing out of the bony tissue

Track 112 in plate 105 may be formed by machining or otherwise removing material from plate 105, or by welding, depositing, or otherwise adding material to plate 105. Track 112 may be a single track disposed longitudinally to capture anchors, or may be two or more tracks 112 disposed laterally to capture anchors. Track 112 may have any cross-sectional profile configured to capture two or more anchors implanted in body tissue. Track 112 may have a depth and cross-sectional profile configured for a selected anchor, such as an angled taper, spherical, or other feature, or a more general cross-sectional profile configured for attachment to a wider range of anchor profiles. Plate 105 is connected to two or more anchors 175 by sliding track 112 to capture the anchor heads 175 on vertebral bodies 170. In some embodiments, track 112 may include a layer (not shown) to provide a selected characteristic, such as a selected friction coefficient, a ratcheting feature for one-way operation, or to provide attachment to a selected anchor profile.

In some embodiments, plate assembly 100 comprises plate 105 may include a continuous top surface that provides a physical barrier to prevent anchors from backing out of bony tissue. In other embodiments, plate 105 may have openings 106 configured small enough to prevent an anchor from backing out of the bony tissue, but large enough to allow access by medical professionals. For example, when anchors are captured in track 112, openings 106 allow a surgeon to visually check the location of the anchors. Openings 106 may also be large enough to insert a tool to allow the surgeon to make adjustments in the depth of penetration of the anchors.

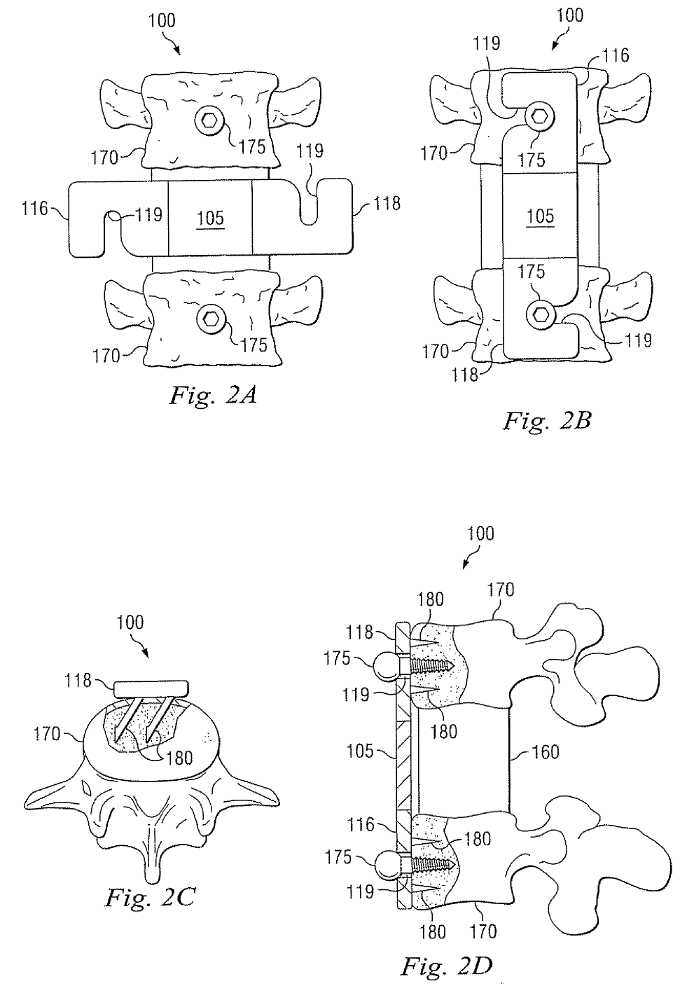

In FIGS. 2A-2D, a plate assembly 100 in accordance with one embodiment of the present disclosure comprises a plate 105 configured for attachment to two anchors, having an attachment feature such as recessed portions 119 on first end 116, and second end 118. Plate assembly 100 may be rotated from the orientation shown in FIG. 2A to the orientation shown in FIG. 2B to securely attach first end 116 to a first anchor 175 and second end 118 to a second anchor 175 by capturing a first anchor 175 in recessed portion 119 on first end 116 and capturing a second anchor 175 in recessed portion 119 of second end 118, and plate 105 may be advantageously configured to prevent anchors 175 from backing out of the bony tissue 170. Plate assembly 100 may be implanted by attaching first end 116 to a first anchor 175 and then rotating second end 118 until a second anchor 175 is captured. In the embodiment shown in FIGS. 2A-2D, first end 116 and second end 118 may be configured to capture both anchors 175 at once by rotating plate assembly 100 in a counter-clockwise direction to capture A recessed portion 119 may be positioned on a first edge of first end 116 and another recessed portion 119 may be positioned on a second edge of second end 118 obverse from the first edge, such that when plate assembly 100 is rotated about a central axis, recessed portions 119 are either both on the leading edge or both on the trailing edge.

First end 116 and second end 118 may be configured for attachment to a selected screw, for example a screw with an angled tapered head, or may be configured for use with a range of anchors. Recessed portions 119 of first end 116 and second end 118 may further have a layer (not shown) for selected characteristics, such as a selected friction coefficient. First end 116 and second end 118 may be identical, or may have individual features tailored for desired functionality. Also, first end 116 and second end 118 may be integral with plate 105 or may be manufactured separately and then mechanically, chemically, or thermally (or some combination) joined to plate 105.

In some embodiments, plate assembly comprises plate 105 having a continuous top surface that provides a physical barrier to prevent anchors from backing out of bony tissue. In other embodiments, plate 105 has openings (such as openings 106 in FIG. 1A) configured small enough to prevent an anchor from backing out of the bony tissue, but large enough to allow access by medical professionals. For example, the openings may be large enough for a surgeon to ensure the plate 105 has captured an anchor 175, or opening may be large enough to allow the surgeon to adjust the depth of penetration of a particular screw. In the embodiment shown in FIG. 2C, holes might not be necessary because anchors can be seen by looking in attachment feature 119. However, in other embodiments in which plate assembly 100 rotates to attach to anchors 175, holes may be desirable for inspection purposes or for access by the surgeon, for example to adjust the depth of an anchor 175 in bony tissue 170.

Furthermore, in situations in which it is desirable to provide spacing between two vertebral bodies, spacer 160 may be fixedly connected to plate 105 and interposed between vertebrae 170. Spacer 160 may further function as a lock out mechanism, or may be rotatably connected to the plate 105 to maintain rotational freedom. Plate 105, first end 116, second end 118, and spacer 160 may also comprise spikes or keels 180 for penetrating a selected bony tissue 170 a desired depth. As an example, spikes 180 on plate 105 may be oriented at some angle relative to plate 105 (as shown in FIG. 2C) and pressed into a bony tissue 170 to prevent plate assembly 100 from counter-rotating and disconnecting.

In FIGS. 3A-B, 4-C, and 5A-B, plate assembly 100 comprises two plates 110 and 120, which may be configured to securely connect by rotating about an axis formed by rotatable connections 141, sliding plates 110 and 120 together using slidable connections 142 or mated connections 143, or a combination to form plate assembly 100. Rotatable connections 141 generally refer to connections in which plates 110 and 120 connect by rotating plates about an axis. Hinges and rivets are two examples of rotatable connectors 141 that permit plate 110 to follow some curvilinear path to connect to plate 120. Slidable connections 142 generally refer to connections in which features on one plate 110 or 120 slide along a rectilinear or curvilinear path or plane in the other plate 120 or 110. Mated connectors 143 generally refer to connections using complementary features, and may include for example tab-recess, one-way, threaded, or quick release connectors.

In FIGS. 3A and 3B, plates 110 and 120 are shown rotatably connected about hinge connector 141 on one end, have a ratcheting connector 143 on the other end, and may be configured with one or more recessed portions 150 configured for capturing a portion of one or more anchors 175 implanted in bony tissue (not shown).

In some embodiments, plates 110 and 120 are symmetric or identical. However, the present disclosure is not so limited. Also, in FIG. 3A and 3B, the interface between plates 110 and 120 is shown generally with rectilinear edges, although the interface may be curvilinear or some combination of rectilinear and curvilinear. Plates 110 and 120 may further comprise spikes or keels to prevent movement after implantation

In some embodiments, plates 110 and 120 connect to form plate assembly 100 having a continuous top surface that provides a physical barrier to prevent anchors from backing out of bony tissue. In other embodiments, plates 110 and/or 120 have openings 106 configured small enough to prevent an anchor from backing out of the bony tissue, but large enough to allow access by medical professionals. For example, opening 106 may be large enough for a surgeon to ensure the plates 110 and 120 have captured a screw, or opening may be large enough to allow the surgeon to insert the tip of a tool to adjust the depth of penetration of an anchor 175 in bony tissue.

In various embodiments, rotatable connector 141 and mated connector 143 may be visible or concealed or located internally or externally in plates 110 and 120, so plates 110 and 120 may appear symmetric or identical. Rotatable (hinged) connector 141 in FIGS. 3A and 3B provides a connection that rotates about an axis, such as an external hinge. In other embodiments, hinge connector 141 is integral to plate 110 or 120 or both. A rivet (not shown) is an example of an internal rotatable connector 141 that plates 110 and 120 may rotate around to capture anchors 175 that have already been implanted in bony tissue.

In some situations, a permanent connection is desirable so the surgeon does not need to assemble the plates, so rotatable connectors 141 may be pre-assembled to permanently connect plates 110 and 120. Advantageously, a permanent rotatable connector 141 may facilitate implantation, for example one-handed operation, and subsequently reduce the likelihood of accidental disconnection.

Also shown in FIGS. 3A and 3B, ratcheting mechanism 143 is one embodiment of a mated connection which may connect plate 110 to plate 120 opposite hinge connector 141 such that one or more anchors 175 are captured when plates 110 and 120 connect. In these embodiments, mated connectors 143 have features on one plate 110 or 120 designed to receive a feature on plate 120 or 110. In FIG. 3A, mated connection 143 is achieved by a ratcheting mechanism that generally has a series of teeth on one plate 110 or 120 to catch a bar, pawl, or protrusion on plate 120 or 110 to capture anchors 175 in bony tissue. Ratcheting connector 143 enables a medical professional to quickly and securely connect plates 110 and 120. Mated connector 143 may provide one-way operation, allow the surgeon to leave a gap between plates 110 and 120, adjust how tight the plates 110 and 120 attach to the anchors, or other parameters based on the desired action.

When plates 110 and 120 are rotated about hinge connector 141 until ratcheting connector 143 is connected, recessed portions 150 along the edge of plates 110 and 120 align to capture anchors 175 to attach plate assembly 100 to the bony tissue. Recessed portion(s) 150 may or may not be evenly spaced, equal depth and equal dimensioned. Recessed portion 150 may be angular, such as having a square, sawtooth, or similar appearance, or may be curved, such as circular, sinusoidal, or similar appearance, or some combination. Those skilled in the art will appreciate that varying the profile of recessed portions 150 may affect how plates 110 and 120 connect, how the anchors are captured, how much movement is allowed once plate assembly 100 is in position, and the capability for plate assembly 100 to provide compression for a graft and other surgical considerations.

Furthermore, in situations in which it is desirable to provide spacing between two vertebral bodies, spacer 160 may be fixedly connected to plates 110 or 120. Spacer 160 may further function as a lock out mechanism, or may be rotatably connected to plates 110 or 120 to maintain rotational freedom. Spacer 160 may incorporate connection features or attachment features.

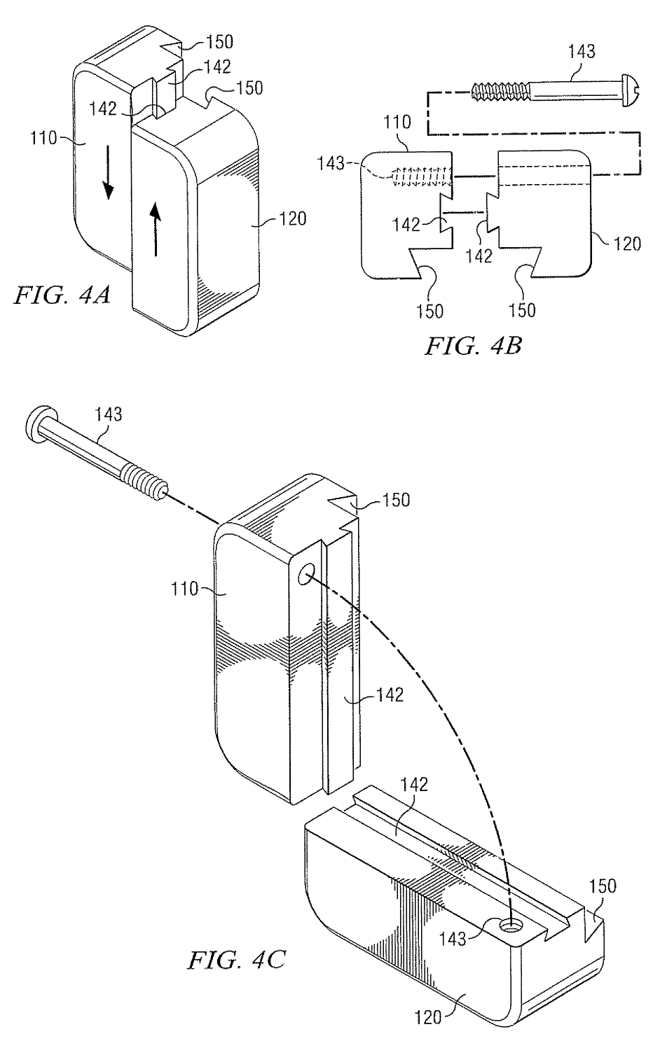

In FIGS. 4A-C, one embodiment of the present disclosure is shown in which plates 110 and 120 may be configured to connect to each other using slidable connector 142 to form plate assembly 100 such that anchors (such as anchors 175 in FIGS. 1A-B, 2A-B, 2D, and 3A-B) are captured in recessed portion 150 to connect plate assembly 100 to two or more bony tissues. Plates 110 and 120 may have a permanent partial connection but completely connect along their full lengths by sliding, or may just have the capability to slide together to permanently connect. In other words, embodiments utilizing a slidable connection may or may not be disconnectable. As plates 110 and 120 slide to connect, the edge of plates 110 and 120 may be configured with one or more recessed portions 150 for capturing anchors. In some embodiments, plate assembly 100 is configured with track or groove 150 to capture anchors to securely attach plate assembly 100 to two or more vertebral bodies. Advantageously, recessed portion 150 still allows some freedom for movement of the vertebral bodies by allowing anchors to move along track 150. One advantage to this type of embodiment is the ability for the medical professional to implant screws anywhere along a plane and the plate assembly 100 can still capture them. Recessed portion 150 may further be tapered such that a screw may slide in the recessed portion 150 until there is a selected contact.

Also shown in FIGS. 4B and 4C is a mated connector 143 utilizing a screw mechanism for connecting plates 110 and 120. In this type of mated connector 143, plates 110 and 120 are connected by turning a screw mechanism 143 on one plate a selected number of rotations to engage corresponding threads in the other plate or until a desired torque value is achieved.

In some embodiments, plates 110 and 120 of plate assembly 100 may be symmetric or identical. However, the present disclosure is not so limited. Also, in FIGS. 4A-C, the interface between plates 110 and 120 is shown generally having rectilinear edges, although the interface may be curvilinear or some combination of rectilinear and curvilinear. Plates 110 and 120 may further comprise spikes or keels to prevent movement after implantation.

In some situations, a permanent connection is desirable, so slidable connectors 142 may permanently connect plates 110 and 120 to capture anchors in recessed portion 150. Advantageously, a permanent slidable connector 142 may facilitate implantation, for example one-handed operation, and subsequently reduce the likelihood of accidental disconnection.

In some embodiments, plates 110 and 120 connect to form plate assembly 100 having a continuous top surface that provides a physical barrier to prevent anchors from backing out of bony tissue In other embodiments, plates 110 and/or 120 have openings (such as openings 106 shown in FIGS. 1A, 1B, 3A and 3B) configured small enough to prevent an anchor from backing out of the bony tissue, but large enough to allow access by medical professionals. For example, opening 106 may be large enough for a surgeon to ensure plates 110 and 120 have captured an anchor, or an opening may be large enough to allow the surgeon to insert a tool to adjust the depth of penetration of an anchor into bony tissue.

Slidable connectors 142 and mated connectors 143 may be visible or concealed or located internally or externally in plates 110 and 120, so plates 110 and 120 may appear symmetric or identical.

Furthermore, in situations in which it is desirable to provide spacing between two vertebral bodies, a spacer (such as spacer 160 of FIG. 2D) may be fixedly connected to plates 110 or 120 for positioning between two vertebral bodies. The spacer may further function as a lock out mechanism, or may be rotatably connected to plates 110 or 120 to maintain rotational freedom. The spacer may incorporate connection features or attachment features.

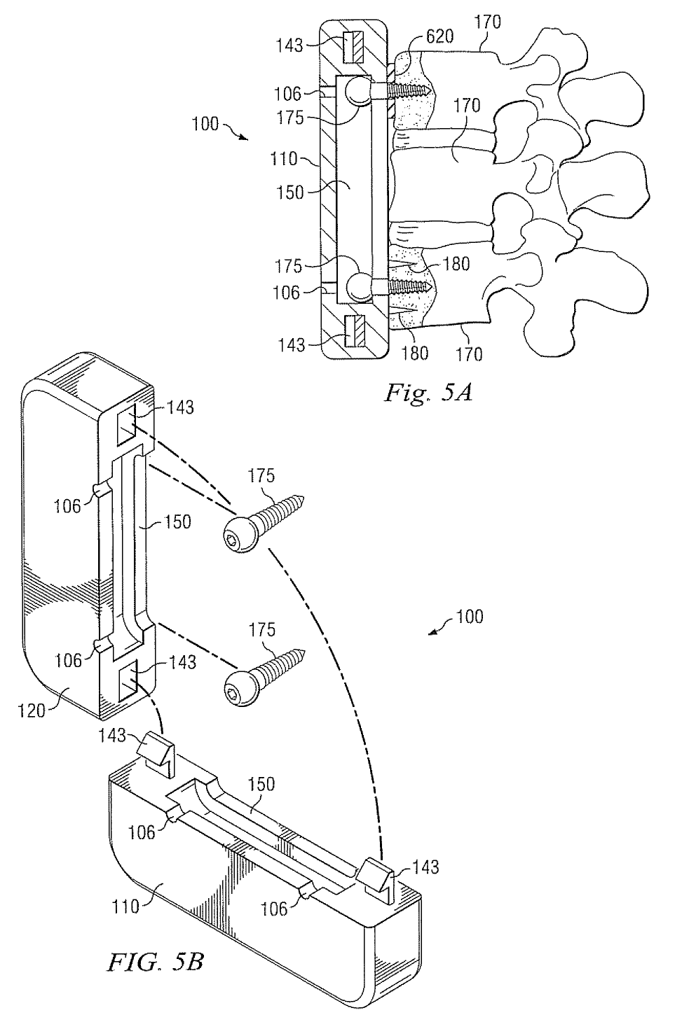

FIGS. 5A and 5B show an embodiment in which a plate assembly 100 comprising plates 110 and 120 that utilize only mated connectors 143 to securely capture anchors 175 in bony tissue 170 such that plate assembly 100 may stabilize movement between bony tissues 170. In this embodiment, plate 110 and 120 are configured to connect one or more recessed portions 150 with a corresponding one or more recessed portions 150 on other plate 110 and 120. Advantageously, plates 110 and 120 may be individually selected for desired characteristics, allowing for more attachment options than prior art plates. As mated connectors 143 on one plate 110 or 120 receive corresponding mated connectors 143 from the other plate 110 or 120, plates 110 and 120 align to capture two or more anchors in recessed portions 150 to securely attach plate assembly 100 to two or more vertebral bodies 170, thus stabilizing movement between the vertebral bodies.

In some embodiments, plates 110 and 120 are symmetric or identical. However, the present disclosure is not so limited. Also, in FIGS. 5A and 5B, the interface between plates 110 and 120 is shown generally with rectilinear edges, although the interface may be curvilinear or some combination of rectilinear and curvilinear. Plates 110 and 120 may further comprise spikes or keels to prevent movement after implantation.

In some embodiments, plates 110 and 120 may connect to form a plate assembly 100 having a continuous top surface that provides a physical barrier to prevent anchors 175 from backing out of bony tissue 170. In other embodiments, plates 110 and/or 120 have openings 106 configured small enough to prevent an anchor from backing out of the bony tissue, but large enough to allow access by medical professionals. For example, opening 106 may be large enough for a surgeon to ensure the plates 110 and 120 have captured a screw, or opening may be large enough to allow the surgeon to insert a tool to adjust the depth of penetration.

In some situations, a permanent connection is desirable, so mated connectors 143 may permanently connect plates 110 and 120. Advantageously, a permanent mated connector 143 may facilitate implantation, for example one-handed operation, and subsequently reduce the likelihood of accidental disconnection.

Furthermore, in situations in which it is desirable to provide spacing between two vertebral bodies, a spacer (such as spacer 160 shown in FIG. 2D) may be fixedly connected to plates 110 or 120 for positioning between two bony tissues. The spacer may further function as a lock out mechanism, or may be rotatably connected to plates 110 or 120 to maintain rotational freedom. The spacer may incorporate connection features or attachment features.

An advantage to all of the embodiments shown and described above is the ability for a plate assembly 100 to attach to a minimum number of screws to stabilize movement between vertebral bodies.

The present disclosure advantageously enables a medical professional to implant single anchors in bony tissues and stabilize movement accordingly. In some embodiments, two or more anchors may be implanted in a single bony tissue for added stability.

In the foregoing specification, the disclosure has been described with reference to specific embodiments. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

What is claimed is:

1. A device for spinal stabilization comprising:
a plate assembly configured to capture a portion of a first anchor located in a first bony tissue and a portion of a second anchor located in a second bony tissue,
wherein the plate assembly is further configured to prevent the first anchor from backing out of the first bony tissue and the second anchor from backing out of the second bony tissue.

2. The device of claim 1, wherein the plate assembly comprises a plate having a track configured to capture a portion of the first anchor and a portion of the second anchor.

3. The device of claim 1, wherein the plate comprises
a first end having a recessed portion on a first edge shaped to receive a portion of the first anchor; and
a second end having a recessed portion on a second edge obverse from the first edge and shaped to receive the portion of the second anchor,
wherein the plate assembly is configured to capture the first and second anchors in the recessed portions by rotating the plate assembly from a first orientation to a second orientation.

4. The device of claim 1, wherein the plate assembly comprises
a first plate configured for selected contact with a plurality of anchors; and
a second plate configured for selected contact with a plurality of anchors, wherein the second plate is configured for secure connection to the first plate to capture the plurality of anchors.

5. The device of claim 4, wherein the first and second plates are configured for slidable connection to capture the plurality of anchors.

6. The device of claim 41 wherein the first and second plates are configured for rotatable connection to capture the plurality of anchors.

7. The device of claim 4, wherein the first and second plates are configured for mated connection to capture the plurality of anchors.

8. The plate assembly of claim 7, wherein the mated connector comprises a ratchet mechanism.

9. The plate assembly of claim 7, wherein the mated connector comprises a screw mechanism.

10. The plate assembly of claim 4, wherein the first plate and the second plate are configured with one or more recessed portions configured for capturing a portion of one or more anchors.

11. The plate assembly of claim 10, wherein the one or more recessed portions comprise a notch.

12. The plate assembly of claim 10, wherein the one or more recessed portions comprise a groove.

13. The plate assembly of claim 1, wherein the plate assembly is manufactured from resorbable material.

14. The plate assembly of claim 1, wherein the plate further comprises a spacer configured for implantation between the first and second bony tissues.

15. The plate of claim 14, wherein the spacer is rotatably attached to the plate.

16. A system for stabilizing movement between two or more bony tissues within a body, comprising:
a first anchor implantable in a first selected bony tissue;
a second anchor implantable in a second selected bony tissue; and
a plate assembly configured to capture a portion of the first anchor and a portion of the second anchor,
wherein the plate assembly is further configured to prevent the first anchor from backing out of the first bony tissue and the second anchor from backing out of the second bony tissue.

17. The system of claim 16, comprising a single anchor implanted in the second bony tissue.

18. The system of claim 16, wherein a portion of one or more of the anchors comprises an angled taper.

19. The system of claim 16, wherein a portion of one or more of the anchors comprises a polyaxial joint.

20. The system of claim 16, wherein a portion of one or more of the anchors comprises a spherical profile.

21. The system of claim 16, wherein a portion of one or more of the anchors comprises a key-style profile.

22. The system of claim 16, wherein one or more anchors further comprise a flange.

23. The system of claim 16, wherein one or more anchors further comprise a spacer.

24. The system of claim 16, wherein the plate assembly further comprises a spacer attached to the plate assembly for positioning between two bony tissues.

25. The system of claim 16, wherein the plate assembly further comprises a spike configured to penetrate into the bony tissue a selected depth.

26. The system of claim 16, wherein the plate assembly comprises
a first end having a recessed portion on a first edge shaped to receive the portion of the first anchor; and
a second end having a recessed portion on a second edge obverse from the first edge and shaped to receive a portion of the second anchor,
wherein the plate is configured to capture the first and second anchors in the recessed portions by rotating between a first orientation and a second orientation.

27. The system of claim 16, wherein the wherein the plate assembly comprises
a first plate configured for selected contact with a plurality of anchors; and
a second plate configured for selected contact with a plurality of anchors,
wherein the second plate is configured for secure connection to the first plate to capture the plurality of anchors

28. The system of claim 27, wherein the first and second plates are configured for slidable connection to capture the plurality of anchors.

29. The system of claim 27, wherein the first and second plates are configured for rotatable connection to capture the plurality of anchors

30. The system of claim 27, wherein the first plate and the second plate are configured with one or more recessed portions configured for capturing a portion of one or more anchors.

31. The system of claim 30, wherein the one or more recessed portions comprises one or more notches.

32. The system of claim 30, wherein the one or more recessed portions comprises a groove.

33. A method for stabilizing movement between bony tissues, comprising the steps of:
implanting a first anchor in a selected location on a first bony tissue;
implanting a second anchor in a selected location on a second bony tissue; and
capturing a portion of the first anchor and a portion of the second anchor, by a plate assembly assembly further configured to prevent the first anchor from backing out of the first bony tissue and the second anchor from backing out of the second bony tissue.

34. The method of claim 33, wherein the bony tissue comprises a vertebral body.

35. The method of claim 33, further comprising the steps of:
implanting a third anchor in a selected location on a third bony tissue; and capturing the first, second and third implanted anchors by the plate assembly.

36. The method of claim 33, further comprising the steps of:
implanting a third anchor in a selected location on the second bony tissue; and
capturing the first, second and third implanted anchors by the plate assembly to stabilize movement between the first and second bony tissues

37. The method of claim 36, wherein the step of capturing the anchors comprises engaging a first plate of the plate assembly to a second plate of the plate assembly.

38. The method of claim 33, wherein the step of securely capturing the two anchors comprises
capturing a first anchor and the second anchor by a single attachment feature.

39. The method of claim 38, wherein the attachment feature comprises a track.

40. The method of claim 33, wherein the first anchor is captured by a first attachment feature and the second anchor is captured by a second attachment feature.

41. The method of claim 33, wherein implanting an anchor further comprises the step of locating an attachment point relative to an anatomical landmark.

42. The method of claim 33, wherein implanting an anchor further comprises the step of locating an attachment point relative to a plane.

43. The method of claim 42, wherein the plane is midline.
